(19) United States
(12) Patent Application Publication
(10) Pub. No.: US 2002/0011250 A1
(43) Pub. Date: Jan. 31, 2002

(54) Procedure for evaluating vestibular dysfunction

(76) Inventors: Kendal L. Stewart, Cedar Park, TX (US); Bridgett D. Wallace, Alexandria, VA (US)

Correspondence Address: AKIN, GUMP, STRAUSS, HAUER & FELD, L.L.P., P.O. BOX 12870, SAN ANTONIO, TX, 78212, US

(21) Appl. No.: 09/909,084

(22) Filed: Jul. 19, 2001

Related U.S. Application Data
(60) Provisional application No. 60/219,788, filed on Jul. 19, 2000.

(51) Int. Cl. A61B 19/00

(52) U.S. Cl. 128/898; 600/301

(57) ABSTRACT

The invention provides standardized guidelines for diagnosis and treatment of central nervous system abnormalities such as concussions, head trauma, memory loss, confusion, or other cognitive dysfunction. An initial assessment is made of the person for loss of consciousness, spinal cord injury, or a secondary injury. The person is asked to perform a plurality of mental and physical exercises. A dynamic visual acuity test is also taken. These tests can be performed on the sideline of an athletic playing field to determine whether or not it is advisable for an athlete to return to play. Next, machine-based hearing, dynamic-vision, and balance tests are administered to the patient. The results of these machine-based tests are transmitted electronically to an online database and evaluated by a physician at an off-site location, who prescribes a treatment regimen designed to improve the patient's vestibular functioning.

This application is based upon and claims priority of U.S. Provisional Patent Application Serial No. 60/219,788 filed Jul. 19, 2000, entitled “Procedure for Evaluating Vestibular Dysfunction.”

### BACKGROUND

### FIELD OF THE INVENTION

The invention relates to the diagnosis of balance disorders, and particularly to a network-based system for remotely evaluating balance-related diagnostic test data.

The brain maintains a sense of spatial orientation and equilibrium by integrating and interpreting a variety of different sensory inputs from the eyes, the inner ear, and the body's somasensory system. The most complex source of spatial cues is the vestibular system of the inner ear. Indeed, studies have shown that the vast majority of athletes complaining of dizziness have a vestibular abnormality.

The inner ear has two types of spatial orientation sensors. Three endolymphatic-fluid-filled semi-circular canals provide sensory information about velocity, rotation, and angular motion. Two otoliths in each inner ear, the utricle and the saccule, sense horizontal and vertical acceleration, respectively. Specialized hair cells in the otoliths and the ampullae (the dilated portions of the semi-circular canals) are displaced by movement in one direction or the other. These displacements are converted to neural signals, which are transmitted by the vestibular nerve to the reticular activating system and lymbic system of the brain. When the head is in a normal upright position, the hair cells in each ear fire equally at a static rate. When the head tilts to the right or to the left, the hair cells in each ear fire at different rates. The central nervous system interprets the inter-aural differences in the firing rate to determine the head's spatial orientation.

The sensory data from the inner ear is complemented by visual cues from the eyes and somatosensory spatial cues from the muscles and skin, which sense pressure and muscle tensioning caused by standing, sitting, or lying down.

Sensory mismatch occurs when the various sensory inputs of the body do not agree with one another. For example, a room on a ship in a stormy sea may look upright at the same time the gravity sensors in the inner ear and pressure sensors in the feet indicate that the room is tilted at an angle. Sensory mismatch can result in nausea, queaziness, and disorientation.

Several different conditions can cause the balance system to malfunction. Labyrinthitis and Vestibular Neuronitis are conditions that describe the inflammation of the inner ear or the vestibular nerve, typically caused by a viral infection. These conditions are characterized by a sudden onset of vertigo which is “spinning” in nature, extreme nausea, and vomiting.

Endolymphatic Hydrops is a condition of the inner ear related to the inability of the ear to regulate its fluid balance, resulting in an episodic buildup of pressure within the balance and hearing organs. This disorder is characterized by spontaneous episodes of vertigo lasting up to several hours, fullness of the ear, “ringing” or noise in the ear, and/or hearing loss. When all of these symptoms are present, the disorder is described as Meniere's Disease. Endolymphatic Hydrops has many causes, including Labyrinthitis, perilymphatic fistula, concussions, noise trauma, autoimmune inner ear disease, and ear surgery.

Benign Paroxysmal Positional Spinning (BPPV) is a condition characterized by the positional onset of vertigo. Brief episodes of vertigo may be caused by lying down, rolling over in bed, or tilting the head back to look up. Canalithiasis, a form of BPPV associated with vertigo lasting only a few seconds, is thought to be caused by misplaced calcium carbonate crystals (otoconia) that have been dislodged from the inner ear and which float freely within the semi-circular canals in response to gravity. Cupulolithiasis, a form of BPPV associated with vertigo lasting several minutes, is caused by misplaced otoconia stuck to the cupula that constantly stimulate the balance sensor.

Perilymphatic fistula is caused by a hole in the inner ear that leaks endolymphatic fluid into the middle ear. Leakage results in brief episodes of vertigo triggered by rapid changes in middle ear pressure, such as that caused by coughing, sneezing, lifting, scuba diving, head trauma, or rapid changes in altitude.

Vestibular migraines are short spells of vertigo, lasting several minutes in duration, accompanied by headaches. This condition is distinguished by the absence of tinnitus or hearing loss.

Vertebribasular Insufficiency (VBI) is a common vascular disorder characterized by decreased blood flow through the vertebrobasilar artery system, which can result in an abrupt onset of vertigo that resolves after several minutes of lying down. Such episodes are accompanied by other VBI symptoms, including visual hallucinations, drop attacks, visual field deficits, diplopia, and headache.

Otoxicity is a condition characterized by damage done to the hearing and balance systems by drugs. Common medications that can permanently damage the inner ear system include Aminoglycoside antibiotics and Cisplatin. Symptoms may include gait unsteadiness, imbalance, and oscillopsia (a bouncing sensation of the horizon).

Other disorders are characterized by gravity sensors in the otolith organs being excessively responsive to internal pressure changes such as that caused by physical activity and to sound. Damage can also be caused by tumors and viral infections that affect the vestibular nerve.

Vestibular abnormalities cause not only the common symptoms of dizziness, spinning, unsteadiness, and nausea, but also cognitive dysfunction, including short-term memory deficits, inability to concentrate or focus on a task, panic attacks, and depression. These cognitive symptoms are caused by the brain's natural compensation mechanisms. To suppress the sensory input from the malfunctioning ear, the brain decreases the relative function of the brain's reticular activating system (RAS) (the portion of the brain responsible for “awareness” or the feeling of being “awake” or “clear”) and limbic system (the portion of the brain responsible for moods and emotions). This adaptive response results in a lowered level of consciousness, manifesting itself in difficulties with cognitive tasks, physical coordination, and short-term memory.

Approximately 11.3 million physician's office visits per year, or five to ten percent of all office visits, are for the complaint of dizziness. A National Institutes of Health study estimates that forty percent of the population over the age of forty have experienced or will experience a “dizziness” disorder during their lifetime. But dizziness affects the young as well. Each year over 450,000 people, mostly young adults, suffer concussions or mild head injuries. Of those requiring short-term hospitalization, a large majority complain of persistent symptoms of dizziness or memory loss though they have been released as “completely normal.”

Unfortunately, many sufferers of dizziness or vertigo disorders do not receive proper diagnosis or therapy. Because the symptoms are vague, and the possible causes many, sufferers are often improperly diagnosed. Worse, sufferers are often treated as “problem patients” or inappropriately referred to psychiatrists and neurologists.

Athletes in contact and high-impact sports experience a high incidence of vestibular abnormalities caused by mild to severe head injuries. Unfortunately, athletes in professional and even college sports are seldom adequately diagnosed for vestibular abnormalities following a jarring collision, blow to the head, or other minor injury before being sent back onto the playing field.

There are a variety of tests for diagnosing balance disorders of the vestibular system. One test is a hearing test. Balance disorders are often accompanied by problems with hearing especially hearing of low frequencies. Another test is a posturography test, in which a person stands on a moving platform and the movement of the ankles and hips are measured. Posturography tests detect the vestibular-spinal reflex, which are manifested in the movements various body muscles make to maintain postural balance and coordination. Posturography tests are useful for analyzing sensory input, thus can isolate the vestibular system to detect pathology.

Another test is an ocular reflex test for testing how quickly and accurately the eyes move to compensate for the movement of the head. When a person without a balance disorder turns his head 30 degrees to the right, the eyes should simultaneously turn 30 degrees to the left. If the eyes turn too little or turn too much, it suggests that the vestibular system may be damaged. The vestibular ocular reflex test is useful in detecting disorders originating in, or affecting, the semi-circular canals of the ear. This is because the brain uses sensory input from the semi-circular canals to generate eye movements that match the velocity of the head movement.

Despite the existence of various balance dysfunction tests, and several treatment centers around the nation, balance disorder treatment is underutilized by the public at large, and athletes in particular.

### SUMMARY OF THE INVENTION

The applicants' research has shown that vestibular abnormalities can be treated with aggressive vestibular therapy, which modifies the central nervous system's interpretation of vestibular input. Such treatment can resolve the secondary effects of RAS inhibition and allow for return to normal performance. Early detection of vestibular abnormalities, followed by early medical intervention, may protect the vestibular apparatus following a mild head injury. Moreover, routine intervention with customized vestibular therapy can help speed return to play.

Accordingly, one aspect of the present invention provides standardized guidelines for diagnosis and treatment of central nervous system abnormalities such as concussions, head trauma, memory loss, confusion, or other cognitive dysfunction. A method is provided for evaluating a person suffering from a central nervous system abnormality. An initial assessment is made of the person for loss of consciousness, spinal cord injury, or a secondary injury. Then the person is asked to perform a plurality of mental exercises, such as describing the time and place, naming the teams in an athletic contest, counting digits backwards, and recalling three words or places. Next, the person is observed while performing a series of physical tests, such as standing straight with the feet together and with the eyes closed, standing straight with the heel of one foot in front of the toes of another and with the eyes closed, running twenty-yards along a straight line with the eyes open, then turning around, making a few steps, and closing the eyes. A dynamic visual acuity test is also taken. Next, machine-based hearing, dynamic-vision, and balance tests are administered to the patient, and these results are evaluated by a physician.

In another aspect of the invention, a method is also provided of administering a program to detect and treat a central nervous system abnormality. A patient completes an on-line consent form and questionnaire, and the results are stored in an on-line database. Later, the patient's vestibular ocular reflex and vestibular spinal reflex are tested with machine-based equipment. The results of these tests are transmitting to the on-line database. An off-site physician retrieves the test results from the on-line database and evaluates the test results to diagnose the abnormality. The physician's diagnosis is transmitted to a patient care specialist, who establishes a course of treatment for the patient. This aspect of the present invention provides off-site “expert” interpretation of balance dysfunction test data and expert guidance through a global network.

Another aspect of the present invention provides widespread access to state-of-the-art diagnostic modalities. Yet another aspect of the present invention provides a platform that integrates a plurality of diagnostic tools, transfers the associated data to a database on the global network, and provides for off-site diagnosis and customization of therapy. Further aspects of the present invention link a regional and/or satellite balance center, the primary care physician, the patient, and a database through a global computer network. Another aspect of the present invention centrally stores clinical and diagnostic information.

One of the advantages of the present invention is that it gives patients, athletes, coaches, physical trainers, and others a more complete picture of how well the vestibular system is interacting with the body and brain. Another advantage is that it aids in accurate evaluation and treatment of the specific sensory abnormality by helping a doctor to distinguish between concussional-type injuries and symptoms related to viral infections, allergies, stress, and other illnesses, producing poor athletic performance. A further advantage of the present information is that it promotes a team approach to working with patients and athletes, by coordinating athletic trainers with physicians and therapists.

A further advantage of the system and procedure of the present invention is that it provides cost-effective screening, diagnosis, and customized therapeutics. A yet further advantage is that a person seeking treatment can apply and set up an appointment for treatment from any personal computer linked to the global network.

Another advantage of the system and procedure of the present invention is that it facilitates early and aggressive vestibular therapy to minimize and compensate vestibular abnormalities. Administered early after a concussion or mild head injury, aggressive vestibular therapy helps the brain to learn to compensate for vestibular abnormalities without suppressing secondary cerebral functions. Even administered long after an injury, the present invention helps restore cognitive abilities impaired by vestibular abnormalities to normal performance.

Another advantage of the system and procedure of the present invention is enhancement of athletic performance. Even minor, subtle vestibular abnormalities, which might be tolerable to an ordinary individual performing ordinary tasks, can impair athletic performance and physical coordination, increasing the risk of further injury. Treatments of athletes in accordance with the present invention enhances the vestibulo-ocular reflex (VOR), increasing the athlete's clarity of vision during rapid eye movement.

The benefits of the present invention redound not only to athletes and concussion sufferers, but also to the elderly and the public at large. Every year, one-third to one-half of the population over age 65 experiences falls, many of which lead to orthopedic injuries, including disabling hip fractures. Many of these falls are due to, or contributed by, underlying inner ear disorders. The present invention's system and procedure for routine and early detection of balance abnormalities significantly reduces the risk of falling for elderly patients.

Moreover, recent clinical experience suggests that some common processing disorders, such as attention deficit disorder, learning disorder, central auditory processing disorder, and visual processing disorder, may merely be symptoms of an underlying inner ear abnormality. Therapeutic regimens administered in accordance with the present invention will improve, if not completely reverse, such central processing disorders.

Other aspects and advantages of the present invention will be more clearly understood after reference to the following detailed description read in conjunction with the drawings.

### BRIEF DESCRIPTION OF THE DRAWINGS

FIG. 1 is a block diagram of one embodiment of a procedure for assessing vestibular dysfunction.

FIG. 2 is a functional flow diagram of one embodiment of a procedure for an in-depth assessment and treatment of the human balance system.

FIG. 3 is a functional flow diagram of one embodiment of an administrative procedure for evaluating an athlete's vestibular functioning and for providing a treatment program.

FIG. 4 is a functional flow diagram of one embodiment of a procedure for sideline evaluation of an athlete who has just suffered a head injury.

### DETAILED DESCRIPTION

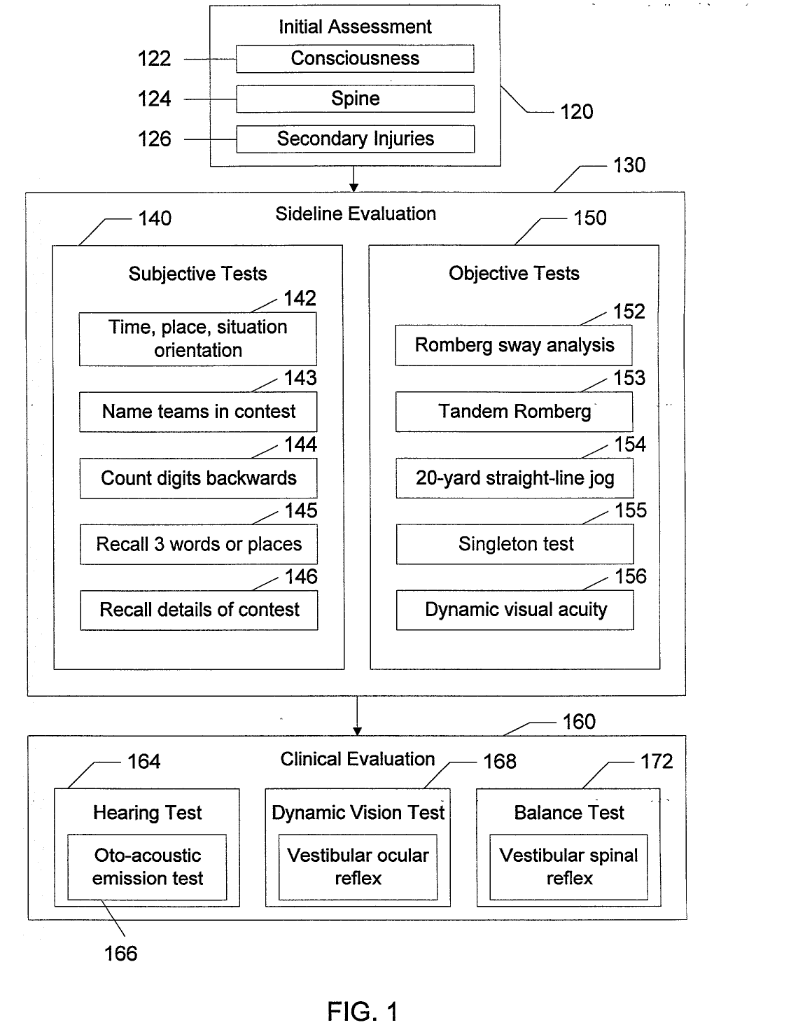

FIG. 1 is a block diagram of one embodiment of a procedure for assessing a central nervous system abnormality (such as a vestibular dysfunction caused by head trauma) carried out in accordance with the present invention. In this exemplary embodiment, an athlete is subjected to three or four batteries of tests. Block 120 represents an initial assessment that is made of the athlete's condition. The initial assessment comprises a plurality of preliminary evaluations denoted by blocks 122, 124, and 126. In block 122, observation is made of, and dialogue is attempted with, the athlete in order to establish a level of consciousness. In block 124, the athlete is examined for injury to the cervical spine. In block 126, the athlete is examined for secondary injuries such as broken bones, sprains and pulled tendons. After this initial assessment is made, and if it is determined that the athlete is conscious and free from spinal and other secondary injuries, the athlete is subjected to another battery of tests. The battery of tests illustrated within block 130 is described as a “sideline evaluation” of the athlete. These tests are so characterized because they can be administered to an athlete on the sidelines of an athletic playing field.

The sideline evaluation comprises, in block 140, a plurality of subjective (i.e., mental) tests, and in block 150, a plurality of objective (i.e., physical) tests. The exemplary embodiment provides five subjective and five objective tests, denoted by blocks 142-146 and 152-156, respectively.

In block 142, the athlete is asked questions to determine his or her orientation to time, place, and situation. In block 143, the athlete is asked to name the teams in the contest in which he or she was participating. In block 144, the athlete is asked to count digits backwards as from 100 to 89. In block 145, the athlete is given a short-term memory test challenging the athlete to recall three words or places. In block 146, the athlete is asked very specific questions regarding the details of the contest such as the score or the quarter in which the teams are playing.

In block 152, the athlete is subjected to a Romberg test, in which the standing athlete is asked to stand upright with feet together and eyes closed. The athlete's sway path and sway area are observed.

In block 153, the athlete is asked to do a Tandem Romberg test for 10 seconds, which requires the athlete to stand straight with the heel of one foot placed in front of the toe of the other foot, while keeping the eyes closed. The amount which the athlete sways while in this posture is observed.

In block 154, the athlete is asked to jog twenty yards in a straight line with his eyes open, then turn around, take a few steps, close the eyes and stand upright. In block 155, the athlete is asked to do the Singleton test, which is five quick steps followed by a 180-degree turn, at which point the athlete closes the eyes, halts, and stands with feet together for 10 seconds.

The physical tests of blocks 152 through 155 represent tests of increasing difficulty. Of course, these tests may be replaced with other physical balance tests without departing from many of the aspects of the present invention. Other gestures the athlete may be asked to perform include standing, standing up and sitting down, reacting to a sudden pushing force in the chest, and turning 360 degrees in one place. In each case, the athlete's responses to positional stress, loss of visual input, and displacement are observed.

In block 156, the athlete's dynamic visual acuity is tested by having the athlete rapidly nod his or her head from side to side and up and down in “no” and “yes” fashion, respectively, while at the same time focusing on an eye chart. The athlete's nodding should approach, but not exceed, 45 degrees from midline in each direction. The test administrator (e.g., the coach) notes the athlete's ability to properly identify alphanumeric symbols on the chart while the head is nodding.

The sideline evaluation tests of block 130 are followed by the more formal clinical tests of block 160. The exemplary embodiment provides three separate tests to quantify damage or injury to each component of the balance system (the vestibular, the visual and somatosensory systems).

In block 164, a hearing test is performed to evaluate the vestibular system. In particular an oto-acoustic emission test is performed. An oto-acoustic emission is a recordable sound generated by the inner ear, either spontaneously or in response to sound stimulation.

In block 168, a dynamic vision test is performed to test the human vestibular ocular reflex. The vestibular ocular reflex maintains stable vision during head movement and is a manifestation of eye-head coordination. The dynamic vision test measures eye movement responsiveness to rapid head motions while the athlete attempts to focus on a static object. An instrument detects and records the movements of the eyes and the head. Examples of machines that test the vestibular ocular reflex include the Vorteq® from Micromedical Technologies, Inc.,™ and the VAT® from Western Systems Research, Inc.™ The resulting data establishes the gain (i.e., the overshoot or undershoot) and the latency of the vestibular ocular reflex in the athlete.

In block 172, a balance test is performed to evaluate the coordination (or mismatch) between the vestibular system and the body's somasensatory system. When operating normally, the vestibular spinal reflex stabilizes the head and the body, and is manifested by movements of the skeleto-muscular system to maintain postural balance. There are a variety of different tests which may be used to test the vestibular spinal reflex, including moving platform posturagraphy which measures the relative movements of the ankles and hips of a person attempting to balance on a moving platform. Other vestibular spinal reflex tests evaluate posture, gait, sway and extremity drift. The vestibular spinal reflex test is useful in detecting dysfunctions involving the otolith organs, which provide inertial gravitational sensory data to the brain for maintaining balance.

The advanced evaluative process of this exemplary embodiment separately quantifies damage to each component of the balance system (the vestibular, visual, and somatosensory systems).

Although this exemplary embodiment provides for three initial assessment tests, five subjective sideline tests, five objective sideline tests, and three clinical evaluation tests, one or more of these particular tests may be omitted or substituted, and one or more other tests may be added, without detracting from many of the aspects of the present invention. Moreover, while the exemplary embodiment utilizes athletic terminology and customized tests for athletes performing in a contest, an equivalent procedure for assessing vestibular dysfunction may be applied to any individual suffering symptoms such as dizziness, nausea, imbalance, or cognitive difficulty without departing from many of the aspects of the present invention.

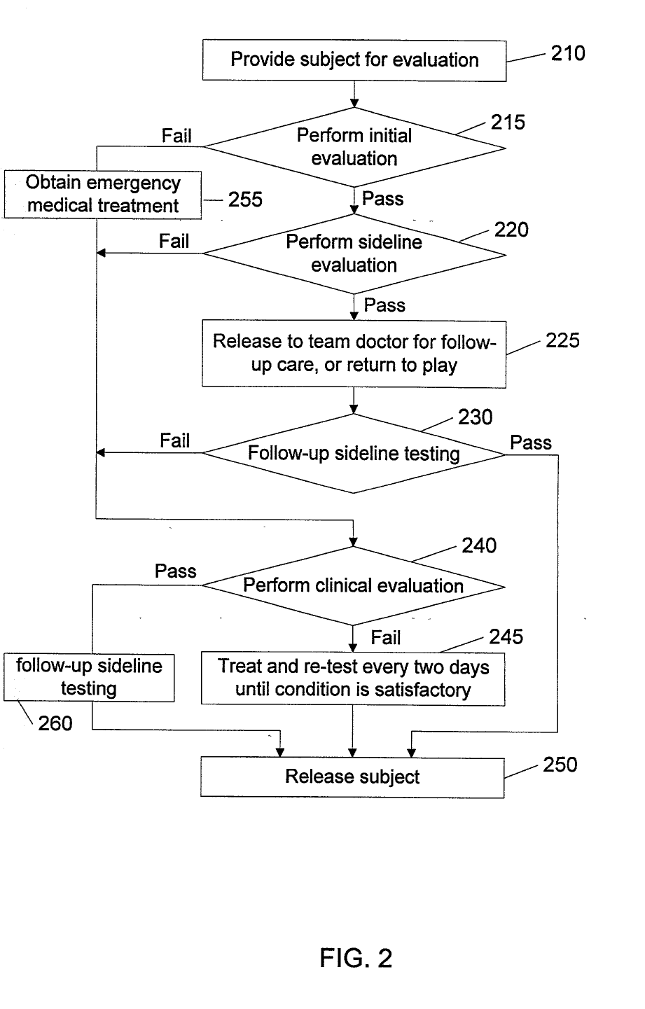

FIG. 2 is a functional flow diagram of one embodiment of a procedure for assessing and treating an athlete's balance system. In block 210, an athlete who has just suffered a minor head injury, or who is due for a routine vestibular condition evaluation, is provided. In block 215, an initial evaluation is performed on the athlete. This initial evaluation may comprise the tests discussed in FIG. 1. This preliminary evaluation may additionally comprise other tests such as pulse rate and blood temperature. If the athlete fails this initial evaluation, in block 255 the athlete is promptly referred for emergency medical treatment. If the athlete passes this initial evaluation, in block 220, various sideline evaluation tests are performed. These sideline evaluation tests include a plurality of subjective and objective tests, such as those illustrated and described in FIG. 1. If the athlete passes these sideline evaluation tests, he or she is released to the team doctor for follow-up care, and is possibly returned to play. In block 230, follow-up sideline testing is performed on the athlete over the next three days. If the athlete passes these tests for three consecutive days, in block 250, the athlete is released.

If the athlete fails either the initial evaluation, the sideline evaluation, or the follow-up sideline testing, then in block 240, a more in-depth clinical evaluation is administered. (If the athlete failed the initial evaluation, then the clinical evaluation is performed only if and after the athlete has been released from emergency medical treatment.) This clinical evaluation comprises a vestibular ocular reflex test and a vestibular spinal reflex test. This clinical evaluation may further comprise other tests, such as a hearing test or an oto-acoustic emission test.

If, in block 240, the athlete passes the clinical evaluation, in block 260, the athlete is given follow-up sideline testing. Else, in block 245, the athlete is treated and retested every two days until his or her vestibular functioning is satisfactory. Once the athlete's vestibular performance is deemed satisfactory, and that performance is confirmed by follow-up sideline testing, then the athlete is released.

Of course, various functions illustrated and described in FIG. 2 may be modified, abbreviated, supplemented and to some extent even rearranged without detracting from many of the aspects of the present invention. Moreover, while this exemplary embodiment illustrates and describes the administration of a procedure to an athlete, an equivalent procedure would be useful to any patient suffering vestibular dysfunction. The procedure of the exemplary embodiment is useful not only for treating severe vestibular dysfunctions, but also for precisely calibrating a person's seemingly satisfactory vestibular system. Combined with the diagnostic procedures of the present invention, various training exercises can help refine and perfect the vestibular function of the body, giving the athlete a competitive edge.

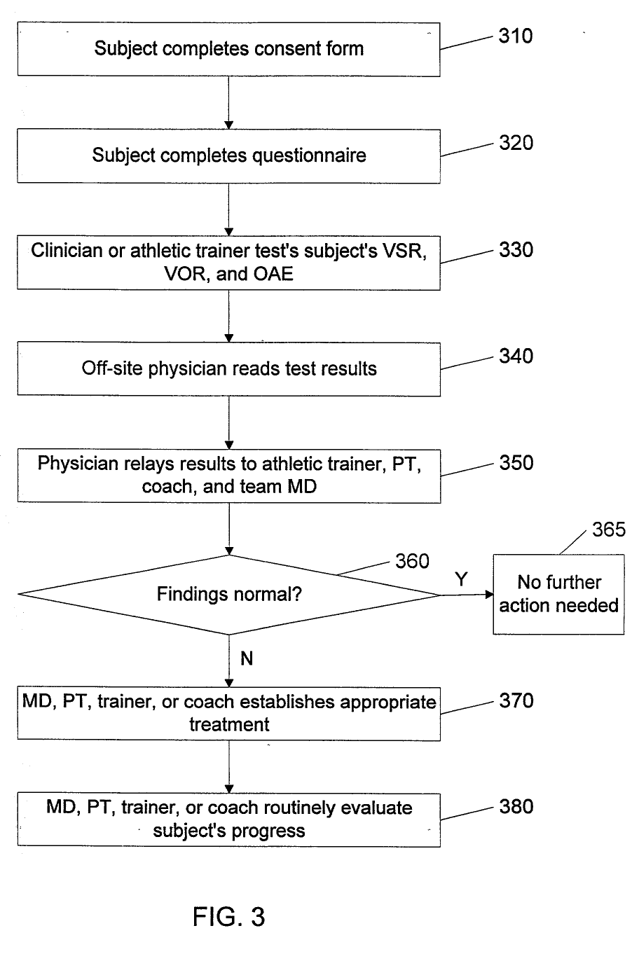

FIG. 3 is a functional flow diagram of one embodiment of an administrative procedure for evaluating an athlete's vestibular functioning and for providing a treatment and research program. In block 310, the athlete completes a form consenting to treatment or consenting to the use of the athlete's results for research purposes. In block 320, the athlete completes a questionnaire. In the exemplary embodiment, the athlete accesses and complete both the consent form and questionnaire on-line from a computer connected to a global computer network.

In block 330, the vestibular ocular and vestibular spinal reflexes of the athlete are tested. In the exemplary embodiment, these tests are performed at a sports health facility or an outpatient clinic by a clinician or athletic trainer. Machinery then records, in an electronically transferrable form, the vestibular ocular and vestibular spinal reflexes of the patient in response to various movements and stimuli.

In block 340, an off-site physician retrieves and evaluates the patient questionnaire answers and the test results. Because the test results and questionnaire answers are recorded in electronically transferrable form, the tests do not have to be performed in the presence or at the location of a physician. Accordingly, in the exemplary embodiment, the questionnaire answers and test results are transmitted electronically over a computer network to a network-accessible database. Then the physician, at a time (within 24 hours of the test) and place convenient to the physician, retrieves and evaluates the test results, prescribing a recommended course of treatment.

In block 350, the off-site physician relays, via the global computer network, his evaluation and treatment recommendation to the athletic trainer, physical therapist, coach, and team doctor. If, in block 360, the test results are normal, then in block 365, no further action is needed. Else, in block 370, the team doctor, physical therapist, athletic trainer, and coach establish an appropriate treatment regimen for the athlete. In block 380, the team doctor, physical therapist, athletic trainer, and coach regularly evaluate the athlete's progress.

Again, as with FIGS. 1 and 2, equivalent embodiments can help most persons, not just athletes, suffering vestibular abnormalities. Other aspects of the exemplary embodiment may be eliminated, modified, substituted, or supplemented, without detracting from many of the aspects of the present invention.

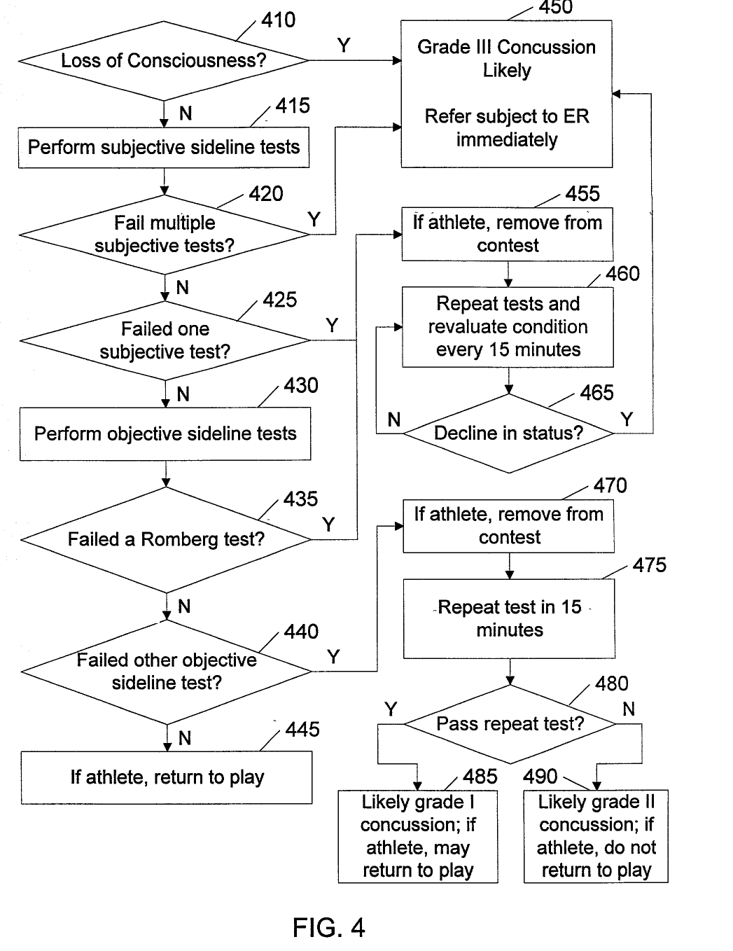

FIG. 4 is a functional flow diagram of one embodiment of a procedure for sideline evaluation of a person suffering from a central nervous abnormality, such as a recent head trauma. This embodiment, as applied to an athlete, permits one to objectively predict an athlete's concussional potential, and to predict whether it is safe for the athlete to return to play. Another benefit is that it allows an athletic trainer to make an accurate and immediate prediction of the athlete's potential for additional injury if he or she is returned to play.

In block 410, the subject is observed for loss of consciousness. If the subject is not fully conscious, then a grade III concussion is likely, and, in block 450, the subject is immediately referred to an emergency room. Else, in block 415, up to five subjective sideline tests are administered. The subjective sideline tests comprise those discussed in FIG. 1 or their equivalents.

If, in block 420, a subject fails two of these subjective sideline tests, then a grade three concussion is likely, and, in block 450, the subject is immediately referred to an emergency room.

If, in block 425, the subject has failed just one of the subjective sideline tests, then, in block 455, the subject is removed from the contest. In block 460, the test is repeated on the subject, and his or her condition reevaluated at regular intervals, or approximately every fifteen minutes. Additionally, the subject's blood pressure and heart rate are evaluated at regular intervals, or approximately every 15 minutes. If, in block 465, the subject's condition declines, then a grade three concussion is likely and the subject is immediately referred to the emergency room.

If the subject has passed all of the subjective tests, then, in block 430, one or more objective sideline tests are administered. If, in block 435, the subject fails a Romberg sway test, then, as illustrated in block 455, the subject is removed from the contest and, in block 460, retested and reevaluated at regular intervals, or approximately every 15 minutes. Again, any decline in the subject's status counsels that the subject be referred to the emergency room immediately.

If, in block 440, the subject passes all of the subjective sideline tests and the Romberg sway tests, but fails one or more of the other objective sideline tests, then, in block 470, the subject is removed from the contest. In block 475, these subjective tests are repeated after a brief interval of time, or approximately fifteen minutes. If, in block 480, there is an improvement in those test results, then it is probable, as illustrated in block 485, that the subject has merely suffered a grade-one concussion. Else, it is likely that the subject has suffered a grade-two concussion, and return to play is not advised.

If the subject passes all of the subjective and objective sideline tests, then the subject is cleared and returned to play in block 445.

The advanced evaluative processes of the present invention integrate a plurality of diagnostic methods that permit an athletic or physical trainer to make a relatively accurate and immediate prediction of a subject's potential for additional injury, and whether the subject can return to play.

While particular embodiments of the invention have been illustrated and described, it will be obvious to those skilled in the art that various changes and modifications may be made without sacrificing the advantages provided by the methods and apparatuses disclosed herein.

We claim:

1. A method of evaluating a person suffering from a central nervous system abnormality, the method comprising:
making an initial assessment of the person's consciousness;
if the person suffers loss of consciousness, then promptly seeking emergency medical attention for the person;
else, assessing the person's ability to perform a plurality of mental exercises;
if the person fails at least two of said plurality of mental exercises, then promptly seeking emergency medical attention for the person;
else if said person fails only one of said plurality of mental exercises, then reevaluating the person's abnormality after a brief period of time, and if the person's abnormality worsens, then promptly seeking emergency medical attention for the person;
if the person passes all of said plurality of mental exercises, then assessing the person's ability to perform a physical balance test;
if the person fails the physical balance test, then re-evaluating the person's abnormality after a brief period of time, and if the person's abnormality worsens, then promptly seeking emergency medical attention for the person.

2. The method of claim 1, further comprising administering a dynamic visual acuity test to the patient.

3. The method of claim 1, wherein the plurality of mental exercises and physical balance tests are performed in close proximity to the sideline of an athletic playing field.

4. The method of claim 1, wherein the plurality of mental exercises comprise:
the person describing the time and place in which the patient is present; and
the person counting digits backwards.

5. The method of claim 1, wherein the physical balance test tests the person's ability to stand and maintain balance while keeping the heel of one of the person's feet directly in front of the toe of another of the person's feet.

6. A method of evaluating a person suffering from a central nervous system abnormality, the method comprising:
making an initial assessment of the person's consciousness;
assessing the person's ability to perform a plurality of mental exercises, said plurality of mental exercises comprising at least one activity selected from the group consisting of describing the time and place in which the person exists, naming teams in a contest, counting digits backwards, recalling specific words, and recalling details of a contest; and
assessing the person's ability to perform a plurality of physical exercises, said plurality of physical exercises comprising at least one activity selected from the group consisting of standing, standing up and sitting down, walking in a straight line, jogging in a straight line, reacting to a sudden pushing force, and turning at least 180 degrees in one place.

7. The method of claim 6, further comprising assessing the ability of the person to identify alphanumeric symbols while the person's head is moving.

8. The method of claim 6, wherein the central nervous system abnormality is a disorder belonging to the group consisting of attention deficit disorders, learning disorders, central auditory processing disorders, and visual processing disorders.

9. The method of claim 6, further comprising testing the person's vestibular ocular reflex.

10. The method of claim 9, wherein the person's vestibular ocular reflex is tested by measuring the responsive movements of the person's eyes to movements of the person's head while the person attempts to focus the person's eyes on a stationary object.

11. The method of claim 6, further comprising testing the person's hearing ability.

12. The method of claim 6, further comprising administering an oto-acoustic emission test to the person.

13. The method of claim 6, further comprising testing the person's vestibular spinal reflex.

14. The method of claim 13, wherein the person's vestibular spinal reflex is tested with a moving platform posturograph machine.

15. The method of claim 13, further comprising testing the person's vestibular ocular reflex.

16. The method of claim 15, further comprising testing the person's hearing.

17. The method of claim 15, further comprising administering an oto-acoustic emission test to the person.

18. A method of administering a program that diagnoses and, if needed, treats a central nervous system abnormality suffered by a person, the method comprising:
assessing the person's ability to perform a plurality of mental exercises, said plurality of mental exercises comprising at least one activity selected from the group consisting of describing the time and place in which the person exists, naming teams in a contest, counting digits backwards, recalling specific words, and recalling details of a contest;
assessing the person's ability to perform a plurality of physical exercises, said plurality of physical exercises comprising at least one activity selected from the group consisting of standing, standing up and sitting down, walking in a straight line, jogging in a straight line, reacting to a sudden pushing force, turning at least 180 degrees in one place, and moving one's head back and forth while identifying alphanumeric symbols;
if the person satisfactorily fails at least one of said plurality of mental and physical exercises, then administering a plurality of machine-based tests that record functional aspects of the person's vestibular, visual, and somatosensory systems;
evaluating the results of said plurality of machine-based tests to design a program of vestibular therapy for the person;
re-administering said plurality of machine-based tests at regular intervals of time, and re-evaluating the results of said machine-based tests, until the person's central nervous system abnormality is resolved.

19. The method of claim 18, wherein the central nervous system abnormality is a disorder belonging to the group consisting of attention deficit disorders, learning disorders, central auditory processing disorders, and visual processing disorders.

20. The method of claim 18, further comprising at least one follow-up diagnosis of the person's ability to perform said plurality of mental and physical exercises.

21. A method of administering a program to detect and treat a central nervous system abnormality, the method comprising:
a patient completing an on-line consent form and questionnaire;
storing patient answers to said questionnaire in an on-line database;
testing the patient's vestibular ocular reflex and vestibular spinal reflex with machine-based equipment, the equipment recording test results in an electronically-transferrable form;
transmitting said test results to said on-line database;
a physician retrieving said test results from the on-line database and evaluating said test results;
the physician diagnosing the abnormality;
transmitting the physician's diagnosis to a patient care specialist;
the patient care specialist establishing a course of treatment for the patient; and
regularly evaluating the patient's progress.

22. The method of claim 21, wherein the machine-based equipment is housed in an onsite outpatient facility, and wherein the physician accesses the on-line database from a location that is offsite from said outpatient facility.

23. The method of claim 21, further comprising administering an oto-acoustic emission test on the patient, recording oto-acoustic emission test results in an electronically-recordable form, and transmitting said oto-acoustic emission test results to said on-line database.

24. The method of claim 21, further comprising:
recording the patient's ability to perform a plurality of mental exercises in the on-line database, said plurality of mental exercises comprising at least one activity selected from the group consisting of describing the time and place in which the person exists, naming teams in a contest, counting digits backwards, recalling specific words, and recalling details of a contest; and
recording the patient's ability to perform a plurality of physical exercises in the on-line database, said plurality of physical exercises comprising at least one activity selected from the group consisting of standing, standing up and sitting down, walking in a straight line, jogging in a straight line, reacting to a sudden pushing force, turning at least 180 degrees in one place, and moving one's head back and forth while identifying alphanumeric symbols.
